(19) United States
(12) Patent Application Publication
(10) Pub. No.: US 2025/0375562 A1
(43) Pub. Date: Dec. 11, 2025

(54) Baby Nasal Aspirator

(71) Applicant: Katie Cabrera, Jurupa Valley, CA (US)

(72) Inventor: Katie Cabrera, Jurupa Valley, CA (US)

(21) Appl. No.: 18/733,916

(22) Filed: Jun. 5, 2024

(51) Int. Cl. A61M 1/00

(52) U.S. Cl. CPC A61M 1/87; A61M 1/741; A61M 2205/8206

(57) ABSTRACT

The Baby Nasal Aspirator is an advanced medical device engineered to alleviate nasal congestion in infants by efficiently removing mucus from their nasal passages. The device comprises a nasal tip, which is gently inserted into the baby's nostril to collect mucus. This mucus is then transported to a detachable mucus trap, ensuring easy disposal and hygienic operation. The mucus trap is connected via tubing to the main aspirator unit, which generates the necessary suction to extract the mucus. A key feature of the Baby Nasal Aspirator is its suction control mechanism, facilitated by a hole port that allows the caregiver to adjust the suction intensity, ensuring a safe and comfortable experience for the infant. The detachable design of the mucus trap simplifies cleaning and maintenance, promoting long-term usability. This device offers a practical solution for managing nasal congestion in infants, contributing to their respiratory comfort and overall health.

### BACKGROUND

### Field of the Invention

The present invention relates to the field of pediatric healthcare, specifically focusing on respiratory care for infants. This device addresses the common issue of nasal congestion in babies, providing a safe and efficient method for mucus removal. It is designed to support parents and caregivers in maintaining infant respiratory health and comfort, enhancing overall pediatric care practices.

### Description of the Related Art

The Baby Nasal Aspirator is an innovative device designed to address the common issue of nasal congestion in infants. Nasal congestion can cause significant discomfort and respiratory difficulties in babies, impacting their ability to feed, sleep, and breathe comfortably. This device aims to provide an efficient, safe, and user-friendly solution for parents and caregivers, ensuring the well-being of infants by effectively clearing nasal passages.

Maintaining clear nasal passages in infants is crucial for their overall health and development. Nasal congestion can lead to breathing difficulties, feeding problems, disturbed sleep, and increased risk of respiratory infections. Effective mucus removal is essential for preventing these issues and ensuring that babies can breathe easily. The Baby Nasal Aspirator addresses this need by providing a reliable tool that parents and caregivers can use to maintain their baby's respiratory health.

Nasal congestion is a common issue among infants, with studies showing that approximately 20% of infants experience significant nasal congestion at some point during their first year of life. This condition is often exacerbated by colds, allergies, and environmental factors. The market for nasal aspirators and related products is growing, with an estimated market size of over $150 million globally in 2023, reflecting the high demand for effective solutions in this category.

Recent advancements in the field of pediatric respiratory care have led to the development of various nasal aspirators, ranging from manual bulb syringes to battery-operated devices. Some of these products include features like adjustable suction levels, multiple nasal tip sizes, and easy-to-clean components. Brands like NoseFrida and Electric Nasal Aspirators have gained popularity for their effectiveness and user-friendly designs. However, these products often come with limitations in terms of suction control, noise levels, and overall ease of use.

By looking at prior art multiple advancements have been seen. For instance, a U.S. Pat. No. 8,696,648B2 Nasal aspirator comprises a resilient bulb and a stem for suction of liquids by vacuum created by compressing the bulb and thereafter gradually releasing the compression of the bulb, where the stem is removable connected to the bulb part, wherein the bulb part comprises a neck portion adopted to interact with a cap portion of the stem part to create a substantially airtight connection there between.

An EP patent 1,474,186B1 relates to Infant nasal aspirator. The Infant nasal aspirators are known in which a hollow nose-piece, that in use communicates with the interior of a nostril, is connected via hollow pipe to a suction device. An aspirator (10) includes a nose-piece having formed therein a hollow chamber containing a sponge-or gauze-like filter (21). The material of the filter (21) absorbs mucus sucked thereinto and thereafter acts as a one way valve preventing backflow of the mucus. Gradual clogging of the filter provides an indication of the remaining useable life of the filter. The filter (21) or the cartridge (14) containing it are disposable.

A CN patent 1,036,91008A relates to Baby nasal aspirator. The invention provides a baby nasal aspirator, and relates to an article for daily use, in particular to a baby product. The baby nasal aspirator comprises a suction head and a rubber pump, wherein the suction head and the rubber pump are connected hermetically. The baby nasal aspirator has the advantages that a part directly contacting with the nasal cavity of a baby is the soft suction head, so that the nasal mucosa of the baby is prevented from being injured; during use, the magnitude of the suction force can be controlled through the rubber pump, so that convenience and safety are realized; after use, the suction head and the rubber pump can be detached, so that the baby nasal aspirator is convenient to clean and is sanitary.

Despite the advancements, many existing nasal aspirators have notable drawbacks. Manual bulb syringes can be difficult to use and may not provide consistent suction, leading to inefficient mucus removal. Battery-operated aspirators often produce excessive noise, which can startle and distress infants. Additionally, many devices lack adjustable suction control, which is crucial for ensuring a safe and comfortable experience for babies with varying degrees of nasal congestion. Cleaning and maintaining these devices can also be challenging, potentially compromising hygiene.

The Baby Nasal Aspirator we have developed is designed to address these shortcomings and provide a superior solution for managing infant nasal congestion. Our device features a soft, flexible nasal tip that ensures gentle insertion and includes a safety stop to prevent over-insertion. The aspirator unit offers adjustable suction control through an innovative hole port mechanism, allowing caregivers to customize the suction intensity. The detachable mucus trap ensures easy cleaning and maintenance, promoting hygiene and long-term usability.

The proposed Baby Nasal Aspirator is uniquely positioned to fill the market gap by combining safety, efficiency, and user-friendliness in one device. Unlike existing products, our aspirator operates with minimal noise, reducing the likelihood of startling the baby during use. The adjustable suction control ensures that the device can be tailored to the specific needs of each infant, providing a more personalized and effective solution. By addressing the common drawbacks of other nasal aspirators, our invention offers a comprehensive tool that meets the needs of both infants and their caregivers, setting a new standard in pediatric respiratory care.

None of the previous inventions and patents, taken either singly or in combination, is seen to describe the instant invention as claimed. Hence, the inventor of the present invention proposes to resolve and surmount existent technical difficulties to eliminate the aforementioned shortcomings of prior art.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

The primary desirable object of the present invention is to provide a novel and improved form of a built-in date tracking system integrated into the lid. This innovative feature allows users to easily monitor the expiration dates of stored food items, promoting efficient food management and reducing waste. With adjustable settings for both month and day, users can conveniently input and update expiration dates as needed, ensuring that food items are consumed before they spoil. This helps users maintain a well-organized and fresh food inventory, leading to healthier eating habits and cost savings.

It is the objective of the invention to provide a safe and effective method for removing mucus from an infant's nasal passages, thereby alleviating congestion and improving respiratory comfort.

It is the objective of the invention to ensure ease of use for parents and caregivers through its user-friendly design, including detachable and easily cleanable components.

It is the objective of the invention to offer adjustable suction control, allowing caregivers to regulate the suction intensity to suit the baby's comfort and safety.

It is the objective of the invention to minimize the risk of injury or discomfort to the baby with a soft, flexible nasal tip that includes a safety stop feature to prevent over-insertion.

It is the objective of the invention to enhance hygiene and reduce the risk of infection by incorporating a detachable mucus trap that can be easily cleaned and sterilized after each use.

It is the objective of the invention to provide a portable solution for nasal congestion, with a compact and lightweight design that can be used at home or on the go.

It is the objective of the invention to reduce parental anxiety and stress by offering a reliable and efficient tool to manage their baby's nasal congestion, ensuring the baby's comfort and health.

It is the objective of the invention to include multiple nasal tip sizes to accommodate different ages and sizes of infants, making the device versatile and adaptable

It is the objective of the invention to operate with minimal noise, ensuring that the baby is not disturbed or frightened during the mucus extraction process.

Thus, it is the objective to improve overall pediatric health care by providing an essential tool that supports better respiratory health in infants, potentially reducing the incidence of respiratory infections and complications. Other aspects, advantages and novel features of the present invention will become apparent from the detailed description of the invention when considered in conjunction with the accompanying drawings.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed invention, and explain various principles and advantages of those embodiments.

FIG. 1 shows the complete view of the proposed assembly incorporating the embodiments of the invention.

FIG. 2 shows one exploded view of the nasal aspirator incorporating the embodiments of the invention.

FIG. 3 shows another exploded view of the nasal aspirator incorporating the embodiments of the invention

FIG. 4 shows the detailed view of nasal tip incorporating the embodiments of the invention.

The apparatus and method components have been represented where appropriate by conventional symbols in the drawings, showing only those specific details

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The Baby Nasal Aspirator is a novel device designed to address nasal congestion in infants by safely and effectively removing mucus from their nasal passages. This device aims to improve respiratory comfort and overall health in babies, making it an essential tool for parents and caregivers. The aspirator consists of several components, including a nasal tip, a detachable mucus trap, a tubing system, and a main aspirator unit. Each component is designed with safety, hygiene, and ease of use in mind, ensuring that the device is both effective and user-friendly.

As per its preferred embodiments, the baby nasal aspirator operates by generating suction through the main aspirator unit, which is connected to a nasal tip via a tubing system. The nasal tip, made of soft, flexible material, is gently inserted into the baby's nostril to extract mucus. The extracted mucus travels through the tubing and is collected in a detachable mucus trap. This design ensures that mucus is efficiently removed and contained, preventing any mess and facilitating easy disposal.

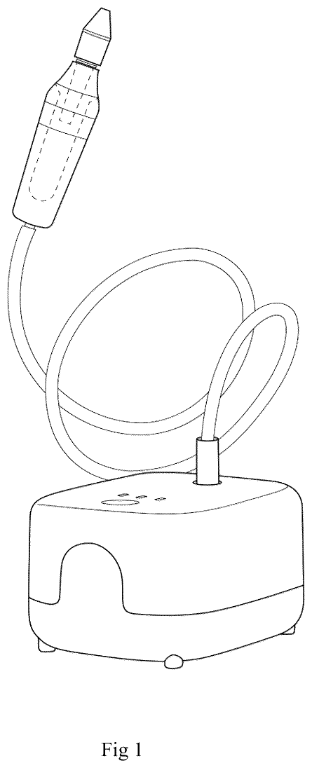

The assembly can be further understood by looking at drawings. FIG. 1 shows the complete assembled baby nasal aspirator. The nasal tip is attached to the tubing, which connects to the main aspirator unit. The tubing allows flexibility and mobility, enabling caregivers to maneuver the nasal tip comfortably while the main unit remains stationary. The main aspirator unit houses the suction mechanism and control features, including an adjustable suction control port. This port allows users to regulate the suction intensity, ensuring a safe and gentle operation suitable for infants.

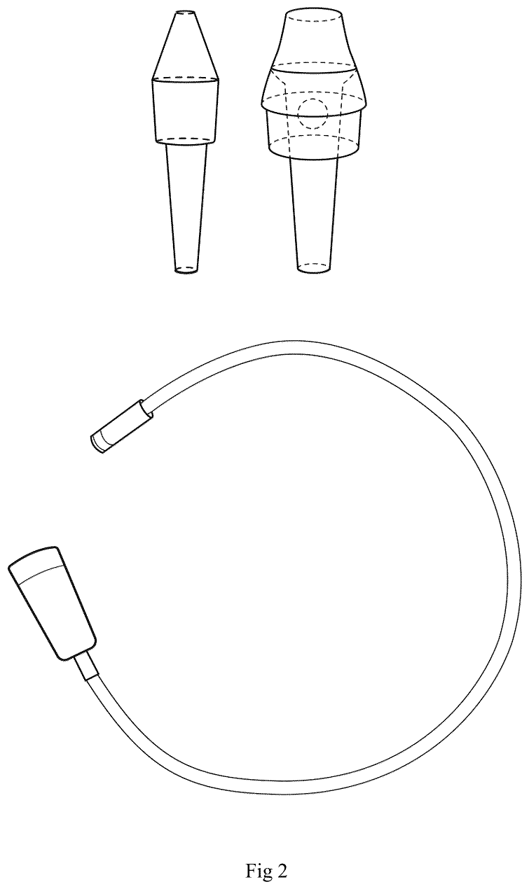

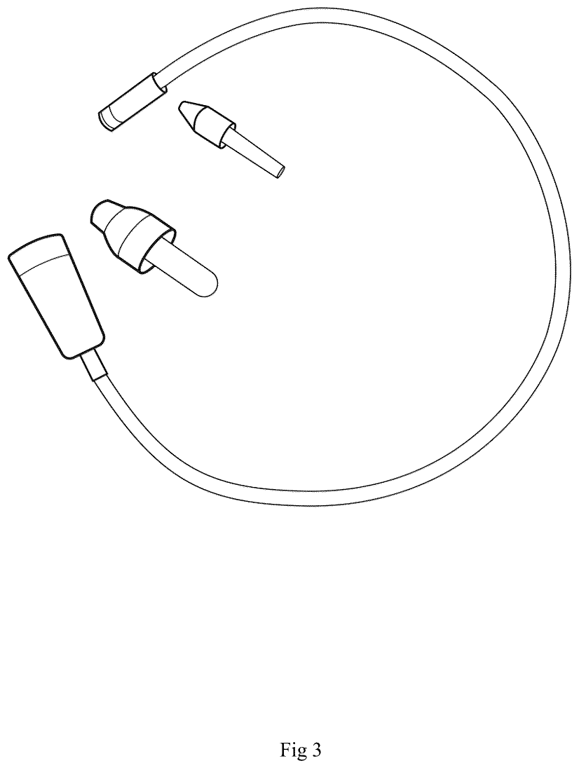

FIGS. 2 and 3 show detailed views of the nasal tips and the tubing system. Two different sizes of nasal tips are depicted, catering to various infant nostril sizes and ages. Each nasal tip is designed with a conical shape for easy insertion and a secure fit. The tubing shown is made of medical-grade silicone, providing durability and maintaining hygiene standards. The detachable nature of these components allows for easy cleaning and replacement, enhancing the device's longevity and usability.

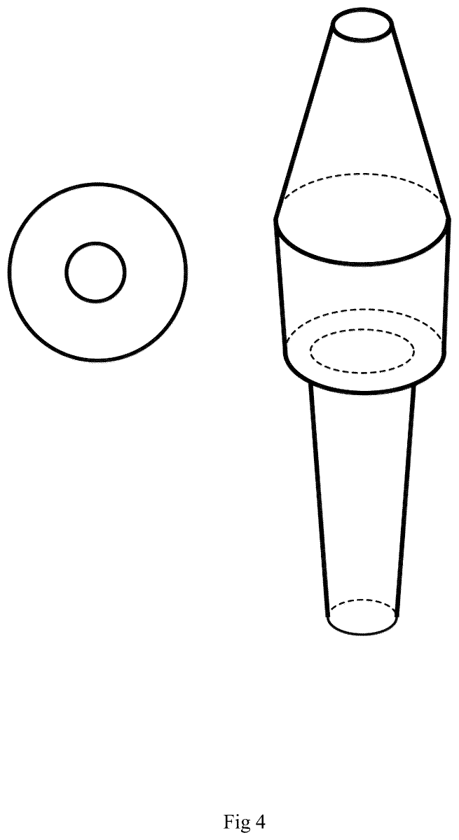

FIG. 4 shows the detailed design of the nasal tip. This component is crucial as it directly interacts with the infant's nasal passages. The nasal tip features a smooth, rounded edge to prevent discomfort or injury during use. Additionally, the nasal tip includes a safety stop feature that prevents over-insertion, ensuring that the device is used safely. The transparent material of the nasal tip allows caregivers to visually monitor the mucus extraction process.

As per its further embodiments, the baby nasal aspirator operates by activating the main aspirator unit, which generates a controlled suction force. The caregiver inserts the nasal tip into the baby's nostril and adjusts the suction intensity using the control port on the main unit. The mucus is then drawn through the nasal tip and tubing, ultimately being collected in the detachable mucus trap. The entire process is designed to be quick, efficient, and comfortable for the baby.

As per its further embodiments, maintaining the baby nasal aspirator is straightforward due to its detachable components. After each use, the nasal tip and mucus trap can be removed, cleaned, and sterilized. The tubing can also be detached and cleaned thoroughly. This ensures that the device remains hygienic and safe for repeated use. The main aspirator unit, which houses the suction mechanism, can be wiped down with a clean, damp cloth.

As per its further embodiments, safety is a paramount consideration in the design of the baby nasal aspirator. The soft, flexible material of the nasal tip minimizes the risk of injury, while the safety stop feature prevents over-insertion. The adjustable suction control allows caregivers to set the appropriate suction level, avoiding excessive force that could harm the baby's delicate nasal tissues. Additionally, the device operates with minimal noise, preventing any distress to the baby during use.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

1. A baby nasal aspirator comprising:
a nasal tip designed to be inserted into a baby's nostril for the purpose of mucus extraction;
a detachable mucus trap for collecting the extracted mucus;
a tubing system connecting the nasal tip to the aspirator unit;
a main aspirator unit generating suction to facilitate mucus removal.

2. The baby nasal aspirator, wherein the suction control mechanism comprises:
a hole port located on the aspirator unit;
an adjustable valve or cover for the hole port, allowing precise regulation of suction intensity.

3. The baby nasal aspirator, wherein the nasal tip is made of soft, flexible material to ensure the comfort and safety of the baby during use.

4. The baby nasal aspirator of claim 1, wherein the mucus trap is transparent to allow visual inspection of the collected mucus.

5. The baby nasal aspirator of claim 1, wherein the tubing is made of medical-grade silicone to ensure durability and hygiene.

6. The baby nasal aspirator of claim 1, wherein the detachable mucus trap includes a secure locking mechanism to prevent accidental spillage.

7. The baby nasal aspirator of claim 1, wherein the main aspirator unit is powered by a rechargeable battery for portable use.

8. The baby nasal aspirator of claim 1, wherein the main aspirator unit includes an automatic shut-off feature to prevent excessive suction.

9. The baby nasal aspirator of claim 1, wherein the entire device is designed to be disassembled easily for thorough cleaning and sterilization.

10. The baby nasal aspirator of claim 1, wherein the nasal tip includes a safety stop feature to prevent over-insertion into the baby's nostril.

11. The baby nasal aspirator of claim 1, wherein the device includes multiple nasal tips of varying sizes to accommodate different ages and sizes of infants.

12. The baby nasal aspirator of claim 1, wherein the aspirator unit operates with minimal noise to avoid disturbing the baby during use.

13. The baby nasal aspirator of claim 2, wherein the adjustable valve or cover is ergonomically designed for easy operation by caregivers.

14. The baby nasal aspirator of claim 2, wherein the suction control mechanism includes marked settings to indicate different levels of suction intensity.

15. The baby nasal aspirator of claim 2, wherein the rechargeable battery has an indicator light to show the battery level.
